SEQUENCE LISTING (6 sequences)

SEQ ID NO: 1 - DNA, 1502 bp, Arabidopsis thaliana
ctaagagttttgctttggtctattgaaagcatgtcaagtcacaaagtcatgagagtttttttttgcttgccaagaaaaaggattggtagaagcagttaataggccgcaggcttggcccctcttttaaagctcaatcttttgctttaaagagttttttgtttagttccaggtgtggaagatgatggttttggaaacttttggtgcaaacaggggagcaaatcttattgctaaaagtgtgacaggagatctgagactgcagtcgtatgtggcaatgggtcctccaagctgcctggtaagagtttttgaggaagaaaggacttgcatctctgtttgttaatgcagtttcgtttcatgggctaaagttctttttgtgaactgatatttgcagtccgttgttgagtttgtttagtgtagggagtttgttttgtctcctgtagtttagaccctcacttttttgtagctatatggctttatcagtgtataaaagccagatgatgagaaaaagaaaaaaaaaattgcagaagaacccttaatttatgacatatataataccactcatgaagaacagttgaaatttctgacaagtaaattgaattccaaccaagtcaaacacgtttattcgagtttcttttttgttttttctcaaagtataataagatgttgaattctgattagctgaaagatgacacgattttattcattgtttttgggtctaccaaaattggaaagtgagtaaataaatggctttctgcgacatttggctttcccatgttactaatttattaaaggaacagttgaacgcaataaataaaaacaaagacatgcactgatcacacaattcctagactcccaaattatcattctaacaggtaaatttcacggtgatatgttaaatatgtagcctgtaggtccaaatacaacaactactttttttgttttttttctattttcattatacatccttattgttatctaattagtaattatcagcattaaagatctgggacactcctgtttaaagctttcatcgactatttgcctattagttgtaacaatcagcctatggttataggtgaatctagactcatataatcaattctgacttaaaccttgtttgaaagtaaagagtgtttctaaacggttacctatagttgtaaacattcatgtctataataaaaacacaccaatatcaatttacacatagccaaataagagtctcaaatgagtcaaaatattaaaactgatttatgaaaacacaaattttataaaaataaatggtgaacaaatgttaagattagtgaaagttcctattttgaaaaacaaatgtctttgaacccaattaatatgccttgtaaaccagtccatagtatcaaactccaaaaaaaaaaagttacttttaatttcaaatatttacataaaattttgcatgcactactctgttcttatataatcttcgtcttcttcttctctgcttgagtcaacaagaaataaacagagttaaa

SEQ ID NO: 2 - DNA, 1676 bp, Arabidopsis thaliana
aatcttcgtcttcttcttctctgcttgagtcaacaagaaataaacagagctaaaatgtttcctctaatctccttttctccaacttctcttgatttcaccttcttcgccattatcatctcaggtttcgttttcatcatcactcgatggaattccaattcaaagaaacgtttaaaccttcctccaggtccgccaggttggccggtggtcggaaacttattccagttcgcgcgctcaggtaaaccgtttttcgaatacgcagaggatctcaagaaaacgtatggcccgatttttacactgagaatggggactcgaacaatgatcatcctctccgacgcaactctagtccacgaagctttgatccaacgcggagctctcttcgcttcacgacccgccgagaatccgacccgaacaatcttcagctgcaacaaattcaccgtcaacgccgctaaatacggtccagtgtggcgatctctgagaagaaacatggttcagaacatgcttagctcaacacgtctcaaggaattcgggaaactgagacaatccgccatggataaactcatcgagaggatcaaatcggaagctagagacaacgacgggctaatctgggttcttaaaaacgcaagattcgctgcgttttgtattctcttggaaatgtgtttcggaatcgagatggatgaagagacaatcgagaagatggatgagattttgaagacggtgttaatgactgttgatccacgaatcgacgattatcttccgattcttgctccgttcttctctaaagagaggaaacgagctctcgaagttcgccgtgaacaagtcgattacgtcgtcggagttatcgagagacgacggagagcgattcagaatccaggatctgataaaacggcgtcgtctttttcatacttagatacactctttgatttaaaaatcgaaggtcgtaaaacgacgccgtctaacgaagagctcgtgacgctctgctctgagtttcttaacggtggtacggatacgacggggacggcgatcgagtgggggatagcgcagctgattgcgaatcctgagattcaatctcggctctacgatgagattaaatcaacggtgggagatgatcgtagggtcgatgagaaagatgtggacaaaatggtctttttacaagcttttgttaaggagcttctccggaaacatccgccgacttatttttctctgacgcatgccgttatggagacgacgacgcttgctggttacgatattcccgccggcgttaacgtcgaggtttatcttccgggtattagtgaggatccgagaatttggaataacccgaaaaagtttgatccggaccggtttatgttgggtaaggaagatgctgacataaccgggattagtggagtaaaaatgattccgtttggtgtgggccgtaggatctgtccaggacttgcaatggcaaccatacacgtccatttgatgcttgcgcggatggttcaggagttcgagtggtgtgctcatccaccaggaagtgagattgatttcgccggaaaattggagtttacggtggtgatgaagaatccgttgagagctatggtcaaaccaaggatttagtcttttattgtttttttgttaagaatattgagattattgtaaggtgtgtttgcttataaacattaaagtacccaaatctacac

SEQ ID NO: 3 - DNA, 54 bp, Artificial sequence
  PCR primer
ggggacaagtttgtacaaaaaagcaggctctaagagttttgctttggtctattg

SEQ ID NO: 4 - DNA, 53 bp, Artificial sequence
  PCR primer
ggggaccactttgtacaagaaagctgggttttagctctgtttatttcttgttg

SEQ ID NO: 5 - DNA, 27 bp, Artificial sequence
  PCR primer
ccccagatctatgtttcctctaatctc

SEQ ID NO: 6 - DNA, 26 bp, Artificial sequence
  PCR primer
ggggggtaccctaaatccttggtttg

(19) United States
(12) Patent Application Publication
(10) Pub. No.: US 2011/0283416 A1
(43) Pub. Date: Nov. 17, 2011

(54) METHODS FOR PLANT FIBER CHARACTERIZATION AND IDENTIFICATION

(75) Inventors: Vincent Sauvephane, Elancourt (FR); Franck Pinot, Westhoffen (FR); Daniele Werck, Souffelweyersheim (FR); Jürgen Ehlting, Victoria (CA)

(73) Assignees: LE CENTRE NATIONAL DE LA RECHERCHE SCIENTIFIQUE, Paris Cedex 16 (FR); BAYER BIOSCIENCE N.V., Gent (BE)

(21) Appl. No.: 13/128,427

(22) PCT Filed: Nov. 11, 2009
(86) PCT No.: PCT/EP2009/008201

§ 371 Date: Jul. 27, 2011

Related U.S. Application Data
(60) Provisional application No. 61/201,654, filed on Dec. 12, 2008.

(30) Foreign Application Priority Data
Nov. 19, 2008 (EP) 08356144.9

(51) Int. Cl. A01H 1/06; A01G 1/00; C12N 5/10; A01H 5/10; C12N 15/82; A01H 5/00

(52) U.S. Cl. 800/278; 435/320.1; 800/298; 800/306; 800/322; 800/320; 800/320.2; 800/320.3; 800/312; 800/320.1; 800/313; 800/314; 435/419; 47/58.1 SE

(57) ABSTRACT

The present invention relates to materials and methods for the expression of a gene of interest preferably in seeds of plants, even more specifically in oilseed plants. In particular, the invention provides an expression cassette for regulating seed-preferential expression in plants.

### FIELD OF THE INVENTION

The present invention relates to materials and methods for the expression of a gene of interest preferably in seeds of plants, even more specifically in oilseed plants. In particular, the invention provides an expression cassette for regulating seed-preferential expression in plants.

### INTRODUCTION TO THE INVENTION

Pants use photosynthetically fixed carbon to support growth and to build up reserve products, such as starch or lipids. Storage oil (triacylglycerol) is a major plant product with great economical importance in human nutrition and as a renewable feedstock for various industrial products and bio-fuels. The world-wide production of vegetable oil is approximately 100 million metric tons in total per year, which mainly consists of soybean, oil palm, rapeseed and sunflower oil. Rapeseed production is increasing world-wide. The crop is mainly used for feed and food, but, increasingly, is being used in bio-diesel production. As a result of the great economic importance of vegetable oils and their expanded use as a renewable feedstock, there is considerable interest in the metabolic engineering of increased end/or modified seed oil content. In seeds of developing oil-seed rape (Brassica napus L.), sucrose is unloaded from the phloem and metabolized to glycolytic intermediates, such as hexose-phosphates, phosphoenolpyruvate and pyruvate, which are subsequently imported into the plastid and used for fatty acid synthesis. Free fatty acids are activated to coenzyme A (CoA) esters, exported from the plastid and used for the stepwise acylation of the glycerol backbone to synthesize triacylglycerol in the endoplasmic reticulum. In the first two steps of triacylglycerol (TAG) assembly, glycerol-3-phosphate (Gly3P) is acylated by Gly3P acyltransferase (GPAT) to lysophosphatidic acid, which is then acylated further by lysophosphatidic acid acyltransferase (LPAT) to phosphatidic acid. This is followed by dephosphorylation of phosphatidic acid by phosphatidic acid phosphohydrolase to release diacylglycerol (DAG), and the final acylation of diacylglycerol by DAG acyltransferase (DAGAT). Final storage of triacylglycerol occurs in endoplasmic reticulum-derived oil bodies.

Modification of oil-producing plants to after and/or improve phenotypic; characteristics (such as productivity or quality) requires the overexpression or down-regulation of endogenous genes or the expression of heterologous genes in plant tissues. Such genetic modification relies on the availability of a means to drive and to control gene expression as required. Indeed, genetic modification relies on the availability and use of suitable promoters which are effective in plants and which regulate gene expression so as to give the desired effect(s) in the transgenic plant. For numerous applications in plant biotechnology a tissue-specific expression profile is advantageous, since beneficial effects of expression in one tissue may have disadvantages in others. Seed-preferential or seed-specific promoters are useful for expressing or down-regulating genes as well as for producing large quantities of protein, and for producing oils or proteins of interest. It is advantageous to have the choice of a variety of different promoters so that the most suitable promoter may be selected for a particular gene, construct, cell, tissue, plant or environment. Moreover, the increasing interest in co-transforming plants with multiple transcription cassettes and the potential problems associated with using common regulatory sequences for these purposes require a variety of promoter sequences. There is, therefore, a great need in the art for the identification of novel sequences that can be used for expression of selected transgenes in economically important plants such as oil-producing plants. It is thus an objective of the present invention to provide new and alternative expression cassettes for seed-preferential or seed-specific expression of transgenes in plants. This objective is solved by the present invention as herein further explained.

Cytochrome P450 mono-oxygenases, which catalyze substrate-, regio- and stereo-specific oxygenation steps in plant metabolism, have evolved to a huge superfamily of enzymes. Plant genome sequencing initiatives recently revealed more than 280 full length genes in Arabidopsis thaliana, 356 in rice and 312 in Populus trichocarpa. However, less than 20% of the coding sequences of the cytochrome P450 mono-oxygenases in the A. thaliana genome have been associated with a specific biochemical function.

### SUMMARY OF THE INVENTION

During our investigation we were interested in the function of “orphan” cytochrome P450 enzymes. One particular enzyme we characterized was the cytochrome P450 mono-oxygenase, CYP77A4, which demonstrated to be a combined fatty acid hydroxylase and epoxidase. Remarkably an expression cassette comprising a chimeric CYP77A4 promoter—nucleic acid fusion proved to be expressed preferably in seeds. The invention described herein in the different embodiments, examples, figures and claims provides seed-preferential promoters and promoter regions comprised in expression cassettes which can be used to direct heterologous gene expression in seeds. In one embodiment the invention provides an expression cassette for regulating seed-preferential expression in plants comprising a promoter linked to a nucleic acid which is heterologous in relation to said promoter and wherein said promoter is selected from the group consisting of (a) SEQ ID NO 1 or a variant thereof, (b) a fragment of at least 50 consecutive bases of SEQ ID NO 1 having promoter activity, (c) a nucleotide sequence with at least 40% identity with SEQ ID NO 1 or the complement thereof and (d) a nucleotide sequence hybridizing under conditions equivalent to hybridization in 7% sodium dodecyl sulfate, 0.5 M NaPO₄, 1 mM EDTA at 50° C. with washing in 2×SSC, 0.1% SDS at 50° C. to a nucleotide sequence depicted in SEQ ID NO 1 or a fragment of at least 50 consecutive bases of SEQ ID NO: 1 or the complement thereof. SEQ ID NO: 1 depicts the nucleotide sequence of the promoter of the cytochrome P450 77A4 gene from A. thaliana.

The expression cassette can direct the expression of a protein, a polypeptide, a peptide, an antisense RNA, a sense RNA or a double-stranded RNA.

In another embodiment the invention provides a recombinant vector comprising an expression cassette as herein described before.

In a further embodiment the invention provides a transformed plant comprising an expression cassette of the invention or a recombinant vector of the invention. The invention further provides transformed plants with the expression cassette or the recombinant vector of the invention. In a particular embodiment the transformed plant is a plant used for oil production. Particular plants are canola, maize, mustard, castor bean, sesame, cotton, linseed, soybean, Arabidopsis, Phaseolus, peanut, alfalfa, wheat, rice, oat, sorghum, rapeseed, rye, sugarcane, safflower, oil palms, flax, sunflower, Brassica campestris, Brassica napus, Brassica juncea, Crambe abyssinica.

Also provided are plant cells comprising an expression cassette of the invention and plant cells comprising a recombinant vector of the invention. In a particular embodiment microspores are provided comprising an expression cassette of the invention and microspores comprising a recombinant vector of the invention.

The invention also provides for a seed generated from the transformed plants wherein the seed comprises an expression cassette of the invention or a vector according to the invention.

Also provided is a method of producing a transformed plant with an expression cassette or a recombinant vector according to the invention, the method comprising providing an expression cassette or a vector of the invention transforming a plant with said expression cassette.

The invention further provides a method for producing a seed enhanced in product of a nucleic acid comprising (a) growing a transformed plant containing the expression vector of the invention, wherein said transformed plant produces said seed and said nucleic acid is transcribed in said seed, and (b) isolating said seed from said transformed plant. In a particular embodiment said nucleic acid encodes for a protein, a polypeptide, a peptide, the expression of an antisense RNA, a sense RNA or a double-stranded RNA.

In yet another embodiment the invention provides the use of SEQ ID NO: 2 or a variant or a functional fragment or a functional homologue with at least 85%, at least 90%, at least 95% identity with SEQ ID NO: 2 for the production of epoxy fatty acids in a host cell. In other words the invention provides a process (or method) for the production of epoxy fatty acids in a host cell comprising a) transforming said host cell with a chimeric construct comprising SEQ ID NO: 2 or a variant or a functional fragment or a functional homologue with at least 85%, at least 90%, at least 95% identity with SEQ ID NO: 2 operably linked to at least one suitable regulatory sequence, b) growing the transformed host cells of step a) and c) determining the presence or the absence of epoxy fatty acids in the transformed cells of step b).

In a particular embodiment said host cell is a plant or plant cell. In yet another particular embodiment said host cell is a yeast cell such as Yarrowia lipolytica, Pichia pastoris, Saccharomyces cerevisiae, a Candida species, a Kluyveromyces species or a Hansenula species.

In yet another embodiment said epoxy fatty acid is a mono-epoxy fatty acid or a bi-epoxy fatty acid, or a tri-epoxy fatty acid or even a tetra-epoxy fatty acid. In a specific embodiment said epoxy fatty acid is vernolic acid.

In yet another embodiment the invention provides a method for the production of 12,13-epoxyoctadeca-9,10-15,16-dienoic acid said method comprising contacting linolenic acid with CYP77A4.

In yet another embodiment the invention provides a chimeric construct comprising SEQ ID NO: 2 or a variant or a functional fragment or a functional homologue with at least 85%, at least 90%, at least 95% identity with SEQ ID NO: 2, operably linked to at least one suitable regulatory sequence. In yet another embodiment the invention provides an isolated host cell comprising a chimeric construct of the present invention.

### BRIEF DESCRIPTION OF FIGURES

FIG. 1: In a quantitative PCR analysis (qPCR) the transcription of cytochrome P450 mono-oxygenase 77A4 (CYP77A4) is quantified for different organs in Arabidopsis thaliana. Error bars on the figure indicate the 95% confidence interval. The values show a seed-enhanced expression in siliques (14-fold higher than roots and 9-fold higher than in the leaves). Z^{ΔΔCt} in the Y-axis shows the relative expression of the CYP77A4 gene.

FIG. 2: In a quantitative PCR analysis the timing of the induction of the transcription of cytochrome P450 CYP77A4 is quantified for 2 plant hormones (methyljasmonate (MeJA) and gibberellins (GA3)) and for mannitol. Water is used as a control. It is observed that mannitol induces a strong expression of CYP77A4 after 33 hours (18-fold induction).

FIG. 3: In a quantitative PCR analysis the timing of the induction of the transcription of cytochrome P450 CYP77A4 is quantified for phenobarbital. Ethanol was used as a control since phenobarbital was solublized in ethanol. It is apparent that Phenobarbital induces a strong expression (12-fold) of CYP77A4 after only 5 hours.

FIG. 4: This figure shows the amount of conversion of different fatty acids (100 μM) with 10 pmole CYP77A4 at 27° C. for 15 minutes. C12:0 (lauric acid) is the optimal substrate of the enzyme (about 35% conversion). C18:0 (stearic acid) is not converted but there is an increased conversion of unsaturated derivatives of C18:0. Indeed, the experiment shows that C18:3 (linolenic acid) is converted for about 20%.

### DETAILED DESCRIPTION OF THE PREFERRED EMBODIMENTS

The present invention provides expression cassettes capable of transcribing a heterologous nucleic acid sequence in a seed, and methods of modifying, producing, and using the same in plants. The present invention also provides compositions, transformed host cells, such as plants, containing an expression cassette with seed-preferred promoters. The nucleotide sequence depicted in SEQ ID NO: 1 represents the nucleotide sequence of the promoter of the cytochrome P450 mono-oxygenase 77A4 gene (herein further designated as CYP77A4) of Arabidopsis thaliana. This gene is located on chromosome V and is described by the GenBank A. thaliana locus At5g04660. Thus the invention provides an expression cassette for regulating seed-preferential expression in plants comprising at least one transcription regulating nucleotide sequence derived from the Arabidopsis thaliana gene described by the GenBank genome locus At5g04660 or its orthologous genes and operably (or “functionally”) linked thereto at least one nucleic acid sequence which is heterologous in relation to said transcription regulating nucleotide sequence.

In one embodiment the invention provides a seed-preferential promoter (or a seed-enhanced promoter which is an equivalent term) having the nucleotide sequence of SEQ ID NO: 1 from nucleotide position 1 to nucleotide position 1502 for use in an expression cassette. In a particular embodiment said seed-preferential promoter has the nucleotide sequence of SEQ ID NO: 1 from nucleotide position 500 to nucleotide position 1502. In yet another particular embodiment said seed-preferential promoter has the nucleotide sequence of SEQ ID NO: 1 from nucleotide position 1000 to nucleotide position 1502. SEQ ID NO: 1 depicts the region upstream (i.e. located 5′ upstream of) from the codon coding for the first amino acid of the CYP77A4 protein. Such a promoter region may be at least about 300 to about 400 to about 500 bp, at least about 1000 bp, at least 1100 bp, at least 1200 bp, at least 1300 bp, at least 1400 bp or at least 1500 bp, upstream of the start codon of the CYP77A4 gene.

The phrases “DNA sequence,” “nucleic acid sequence,” and “nucleic acid molecule” refer to a physical structure comprising an orderly arrangement of nucleotides. The DNA sequence or nucleotide sequence may be contained within a larger nucleotide molecule, vector, or the like. In addition, the orderly arrangement of nucleic acids in these sequences may be depicted in the form of a sequence listing, figure, table, electronic medium, or the like. The term “expression” refers to the transcription of a gene to produce the corresponding RNA. In a particular embodiment said RNA is mRNA and translation of this mRNA produces the corresponding gene product (i.e., a peptide, polypeptide, or protein). In another particular embodiment the heterologous nucleic acid, operably linked to the promoters of the invention, may also code for antisense RNA, sense RNA, double stranded RNA or synthetic microRNA molecules, according to rules well known in the art, to down-regulate the expression of other genes comprised within the seed or even of genes present within a pathogen or pest that feeds upon the seeds of the transgenic plant.

The term “heterologous” refers to the relationship between two or more nucleic acid or protein sequences that are derived from different sources. For example, a promoter is heterologous with respect to a coding sequence if such a combination is not normally found in nature. In addition, a particular sequence may be “heterologous” with respect to a cell or organism into which it is inserted (i.e., does not naturally occur in that particular cell or organism).

The term “chimeric gene” refers to any gene that contains: a) DNA sequences, including regulatory and coding sequences that are not found together in nature, or b) sequences encoding parts of proteins not naturally adjoined, or c) parts of promoters that are not naturally adjoined. Accordingly, a chimeric gene may comprise regulatory sequences and coding sequences that are derived from different sources, or comprise regulatory sequences, and coding sequences derived from the same source, but arranged in a manner different from that found in nature. In the current invention a “homologous” gene or polynucleotide or polypeptide refers to a gene or polynucleotide or polypeptide that shares sequence similarity with the gene or polynucleotide or polypeptide of interest.

“Seed-specific” expression (or transcription which is equivalent) in the context of this invention means the transcription of a nucleic acid sequence by a promoter (or a transcription regulating element) in a way that transcription of said nucleic acid sequence in seeds contribute to more than 90%, preferably more than 95%, more preferably more than 99% of the entire quantity of the RNA transcribed from said nucleic acid sequence in the entire plant during any of its developmental stage.

“Seed-preferential” expression (or transcription which is equivalent) in the context of this invention means the transcription of a nucleic acid sequence by a transcription regulating element in a way that transcription of said nucleic acid sequence in seeds contributes to more than 50%, preferably more than 60%, more preferably more than 70%, even more preferably more than 80% of the entire quantity of the RNA transcribed from said nucleic add sequence in the entire plant during any of its developmental stages. The term “seed-enhanced” is equivalent to the term “seed-preferential”.

The transcription sequences identified herein are found to mediate embryo-preferred or embryo-specific expression. A seed is representing an embryo in its shell, or the embryo is a part of the seed. In the context of this invention, the term seed-preferential also means embryo-preferential, and seed-specific means embryo-specific. These terms can be used interchangeable herein.

“Seed” means a seed of a plant in any stage of its development i.e. starting from the fusion of pollen and oocyte, continuing over the embryo stage and the stage of the dormant seed, until the germinating seed, ending with early seedling organs, as e.g. cotyledons and hypocotyls. “Microspore”—in seed plants—corresponds to the developing pollen grain at the uninucleate stage.

The phrase “operably linked” refers to the functional spatial arrangement of two or more nucleic acid regions or nucleic acid sequences. For example, a promoter region may be positioned relative to a nucleic acid sequence such that transcription of a nucleic acid sequence is directed by the promoter region. Thus, a promoter region is “operably linked” to the nucleic acid sequence. “Functionally linked” is an equivalent term.

As used herein, “promoter” means a region of DNA sequence that is essential for the initiation of transcription of DNA, resulting in the generation of an RNA molecule that is complimentary to the transcribed DNA; this region may also be referred to as a “5′ regulatory region.” Promoters are usually located upstream of the coding sequence to be transcribed and have regions that act as binding sites for RNA polymerase II and other proteins such as transcription factors (trans-acting protein factors that regulate transcription) to initiate transcription of an operably linked gene. Promoters may themselves contain sub-elements (i.e. promoter motifs) such as cis-elements or enhancer domains that regulate the transcription of operably linked genes. The promoters of this invention may be altered to contain “enhancer DNA” to assist in elevating gene expression. As is known in the art, certain DNA elements can be used to enhance the transcription of DNA. These enhancers often are found 5′ to the start of transcription in a promoter that functions in eukaryotic cells, but can often be inserted upstream (5′) or downstream (3′) to the coding sequence. In some instances, these 5′ enhancer DNA elements are introns. Among the introns that are useful as enhancer DNA are the 5′ introns from the rice actin 1 gene (see U.S. Pat. No. 5,641,876), the rice actin 2 gene, the maize alcohol dehydrogenase gene, the maize heat shock protein 70 gene (see U.S. Pat. No. 5,593,874), the maize shrunken 1 gene, the light sensitive 1 gene of Solanum tuberosum, and the heat shock protein 70 gene of Petunia hybrida (see U.S. Pat. No. 5,659,122). Thus, as contemplated herein, a promoter or promoter region includes variations of promoters derived by inserting or deleting regulatory regions, subjecting the promoter to random or site-directed mutagenesis, etc. The activity or strength of a promoter may be measured in terms of the amounts of RNA it produces, or the amount of protein accumulation in a cell or tissue, relative to a promoter whose transcriptional activity has been previously assessed.

Confirmation of promoter activity for a functional promoter fragment in seed may be determined by those skilled in the art, for example using a promoter-reporter construct comprising the genomic sequence operably linked to a beta-glucuronidase (GUS) reporter gene as herein further explained. The seed-preferential expression capacity of the identified or generated fragments of the promoters of the invention can be conveniently tested by operably linking such DNA molecules to a nucleotide sequence encoding an easy scorable marker, e.g. a beta-glucuronidase gene, introducing such a chimeric gene into a plant and analyzing the expression pattern of the marker in seeds as compared with the expression pattern of the marker in other parts of the plant. Other candidates for a marker (or a reporter gene) are chloramphenicol acetyl transferase (CAT) and proteins with fluorescent properties, such as green fluorescent protein (GFP) from Aequora victoria. To define a minimal promoter region, a DNA segment representing the promoter region is removed from the 5′ region of the gene of interest and operably linked to the coding sequence of a marker (reporter) gene by recombinant DNA techniques well known to the art. The reporter gene is operably linked downstream of the promoter, so that transcripts initiating at the promoter proceed through the reporter gene. Reporter genes generally encode proteins, which are easily measured, including, but not limited to, chloramphenicol acetyl transferase (CAT), beta-glucuronidase (GUS), green fluorescent protein (GFP), beta-galactosidase (beta-GAL), and luciferase. The expression cassette containing the reporter gene under the control of the promoter can be introduced into an appropriate cell type by transfection techniques well known to the art. To assay for the reporter protein, cell lysates are prepared and appropriate assays, which are well known in the art, for the reporter protein are performed. For example, if CAT were the reporter gene of choice, the lysates from cells transfected with constructs containing CAT under the control of a promoter under study are mixed with iso-topically labeled chloramphenicol and acetyl-coenzyme A (acetyl-CoA). The CAT enzyme transfers the acetyl group from acetyl-CoA to the 2- or 3-position of chloramphenicol. The reaction is monitored by thin-layer chromatography, which separates acetylated chloramphenyicol from unreacted material. The reaction products are then visualized by autoradiography. The level of enzyme activity corresponds to the amount of enzyme that was made, which in turn reveals the level of expression and the seed-preferential functionality from the promoter or promoter fragment of interest. This level of expression can also be compared to other promoters to determine the relative strength of the promoter under study, Once activity and functionality is confirmed, additional mutational and/or deletion analyses may be employed to determine the minimal region and/or sequences required to initiate transcription. Thus, sequences can be deleted at the 5′ end of the promoter region and/or at the 3′ end of the promoter region, and nucleotide substitutions introduced. These constructs are then again introduced in cells and their activity and/or functionality determined.

Instead of measuring the activity of a reporter enzyme, the transcriptional promoter activity (and functionality) can also be determined by measuring the level of RNA that is produced. This level of RNA, such as mRNA, can be measured either at a single time point or at multiple time points and as such the fold increase can be average fold increase or an extrapolated value derived from experimentally measured values. As it is a comparison of levels, any method that measures mRNA levels can be used. In a preferred aspect, the tissue or organs compared are a seed or seed tissue with a leaf or leaf tissue. In another preferred aspect, multiple tissues or organs are compared. A preferred multiple comparison is a seed or seed tissue compared with 2, 3, 4, or more tissues or organs selected from the group consisting of floral tissue, floral apex, pollen, leaf, embryo, shoot, leaf primordia, shoot apex, root, root tip, vascular tissue and cotyledon. As used herein, examples of plant organs are seed, leaf, root, etc. and example of tissues are leaf primordia, shoot apex, vascular tissue, etc. The activity or strength of a promoter may be measured in terms of the amount of mRNA or protein accumulation it specifically produces, relative to the total amount of mRNA or protein. The promoter preferably expresses an operably linked nucleic acid sequence at a level greater than about 2.5%, more preferably greater than about 5, 6, 7, 8, or about 9%, even more preferably greater than about 10, 11, 12, 13, 14, 15, 16, 17, 18, or about 19%, and most preferably greater than about 20% of the total mRNA. Alternatively, the activity or strength of a promoter may be expressed relative to a well-characterized promoter (for which transcriptional activity was previously assessed).

It will herein further be clear that equivalent CYP77A4 promoters can be isolated from other plants. To this end, orthologous promoter fragments may be isolated from other plants using SEQ ID NO: 1 or a functional fragment having at least 50 consecutive nucleotides thereof as a probe and identifying nucleotide sequences from these other plants which hybridize under the elected hybridization conditions. By way of example, a promoter of the invention may be used to screen a genomic library of a crop or plant of interest to isolate corresponding promoter sequences according to techniques well known in the art. Thus, a promoter sequence of the invention may be used as a probe for hybridization with a genomic library under medium to high stringency conditions.

The term “hybridization” refers to the ability of a first strand of nucleic acid to join with a second strand via hydrogen bond base pairing when the two nucleic acid strands have sufficient sequence identity. Hybridization occurs when the two nucleic acid molecules anneal to one another under appropriate conditions. Nucleic acid hybridization is a technique well known to those of skill in the art of DNA manipulation. The hybridization property of a given pair of nucleic acids is an indication of their similarity or identity. Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence. “Stringent hybridization conditions” and “stringent hybridization wash conditions” in the context of nucleic acid hybridization experiments such as Southern and Northern hybridization are sequence dependent, and are different under different environmental parameters. An example of highly stringent wash conditions is 0.15 M NaCl at 72° C. for about 15 minutes. An example of stringent wash conditions is a 0.2×SSC wash at 65° C. for 15 minutes, Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4 to 6×SSC at 40° C. for 15 minutes. For short probes (e.g., about 10 to 50 nucleotides), stringent conditions typically involve salt concentrations of less than about 1.5 M, more preferably about 0.01 to 1.0 M, Na on concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code. Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SOS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. The following are examples of sets of hybridization/wash conditions that may be used to clone orthologous nucleotide sequences that are substantially identical to reference nucleotide sequences of the present invention: a reference nucleotide sequence preferably hybridizes to the reference nucleotide sequence in 7% sodium dodecyl sulfate (SOS), 0.5 M NaPO₄, 1 mM EDTA at 50° C. with washing in 2×SSC, 0.1% SDS at 50° C., more desirably in 7% sodium dodecyl sulfate (SDS). 0.5 M NaPO₄, 1 mM EDTA at 50° C. with washing in 1×SSC, 0.1% SOS at 50° C., more desirably still in 7% sodium dodecyl sulfate (SOS), 0.5 M NaPO₄, 1 mM EDTA at 50° C. with washing in 0.5×SSC, 0.1% SDS at 50° C., even more desirably still in 7% sodium dodecyl sulfate (SDS). 0.5 M NaPO₄, 1 mM EDTA at 50° C. with washing in 0.1×SSC, 0.1% SDS at 50° C.

In another embodiment of the present invention seed-preferential promoters are provided which comprise a nucleotide sequence having at least 40%, at least 50%, or at least 60%, or at least 70%, or at least 80%, or at least 90%, or at least 95% sequence identity to the herein described promoters and promoter regions. The term “variant” with respect to the transcription regulating nucleotide sequence SEQ ID NO: 1 of the invention is intended to mean substantially similar sequences. Naturally occurring allelic variants such as these can be identified with the use of well-known molecular biology techniques, as, for example, with polymerase chain reaction (PCR) and hybridization techniques as herein outlined before. Variant nucleotide sequences also include synthetically derived nucleotide sequences, such as those generated, for example, by using site-directed mutagenesis of SEQ ID NO: 1. Generally, nucleotide sequence variants of the invention will have at least 40%, 50%, 60%, to 70%, e.g., preferably 71%, 72%, 73%, 74%, 75%, 76%, 77%, 78%, to 79%, generally at least 80%, e.g., 81% to 84%, at least 85%, e.g., 86%, 87%, 88%, 89%, 90%, 91%, 92%, 93%, 94%, 95%, 96%, 97%, to 98% and 99% nucleotide sequence identity to the native (wild type or endogenous) nucleotide sequence. Derivatives of the DNA molecules disclosed herein may include, but are not limited to, deletions of sequence, single or multiple point mutations, alterations at a particular restriction enzyme site, addition of functional elements, or other means of molecular modification which may enhance, or otherwise alter promoter expression. Techniques for obtaining such derivatives are well-known in the art (see, for example, J. F. Sambrook, D. W. Russell, and N. Irwin (2000) Molecular Cloning: A Laboratory Manual, 3^{rd} edition Volumes 1, 2, and 3. Cold Spring Harbor Laboratory Press). For example, one of ordinary skill in the art may delimit the functional elements within the promoters disclosed herein and delete any non-essential elements. Functional elements may be modified or combined to increase the utility or expression of the sequences of the invention for any particular application. Those of skill in the art are familiar with the standard resource materials that describe specific conditions and procedures for the construction, manipulation, and isolation of macromolecules (e.g., DNA molecules, plasmids, etc.), as well as the generation of recombinant organisms and the screening and isolation of DNA molecules. As used herein, the term “percent sequence identity” refers to the percentage of identical nucleotides between two segments of a window of optimally aligned DNA. Optimal alignment of sequences for aligning a comparison window are well-known to those skilled in the art and may be conducted by tools such as the local homology algorithm of Smith and Waterman (Waterman, M. S. introduction to Computational Biology: Maps, sequences and genomes. Chapman & Hail. London (1995), the homology alignment algorithm of Needleman and Wunsch (J. Mol, Biol., 48:443-453 (1970), the search for similarity method of Pearson and Lipman (Proc. Natl. Acad. Sci., 85:2444 (1988), and preferably by computerized implementations of these algorithms such as GAP, BESTFIT, FASTA, and TFASTA available as part of the GCG (Registered Trade Mark), Wisconsin Package (Registered Trade Mark from Accelrys Inc., San Diego, Calif.). An “identity fraction” for aligned segments of a test sequence and a reference sequence is the number of identical components that are shared by the two aligned sequences divided by the total number of components in the reference sequence segment, i.e., the entire reference sequence or a smaller defined part of the reference sequence. Percent sequence identity is represented as the identity fraction times 100. The comparison of one or more DNA sequences may be to a full-length DNA sequence or a portion thereof, or to a longer DNA sequence.

The promoters of the present invention may be operably linked to a nucleic acid sequence that is heterologous with respect to the promoter. The nucleic acid sequence may generally be any nucleic acid sequence for which an increased level or altered level (e.g. in a different organ) of transcription is desired. The nucleic acid sequence can for example encode a polypeptide that is suitable for incorporation into the diet of a human or an animal or can provide some other agricultural or industrial important feature. Suitable heterologous nucleic acid sequences include, without limitation, those encoding seed storage proteins, fatty acid pathway enzymes, epoxidases, hydroxylases, cytochrome P450 mono-oxygenases, desaturases, tocopherol biosynthetic enzymes, amino acid biosynthetic enzymes, steroid pathway enzymes, carotenoid pathway enzymes and starch branching enzymes.

In another embodiment the invention provides a vector, in particular a recombinant vector comprising an expression cassette of the invention. A “recombinant vector” refers to any agent such as a plasmid, cosmid, virus, autonomously replicating sequence, phage, or linear single-stranded, circular single-stranded, linear double-stranded, or circular double-stranded DNA or RNA nucleotide sequence. The recombinant vector may be derived from any source and is capable of genomic integration or autonomous replication. Thus, any of the promoters and heterologous nucleic acid sequences described above may be provided in a recombinant vector. A recombinant vector typically comprises, in a 5′ to 3′ orientation: a promoter to direct the transcription of a nucleic acid sequence and a nucleic acid sequence. The recombinant vector may further comprise a 3′ transcriptional terminator, a 3′ polyadenylation signal, other untranslated nucleic acid sequences, transit and targeting nucleic acid sequences, selectable markers, enhancers, and operators, as desired. The wording “5′ UTR” refers to the untranslated region of DNA upstream, or 5′ of the coding region of a gene and “3′ UTR” refers to the untranslated region of DNA downstream, or 3′ of the coding region of a gene. Means for preparing recombinant vectors are well known in the art. Methods for making recombinant vectors particularly suited to plant transformation are described in U.S. Pat. No. 4,971,908, U.S. Pat. No. 4,940,835, U.S. Pat. No. 4,769,061 and U.S. Pat. No. 4,757,011. Typical vectors useful for expression of nucleic acids in higher plants are well known in the art and include vectors derived from the tumor-inducing (Ti) plasmid of Agrobacterium tumefaciens. One or more additional promoters may also be provided in the recombinant vector. These promoters may be operably linked, for example, without limitation, to any of the nucleic acid sequences described above. Alternatively, the promoters may be operably linked to other nucleic acid sequences, such as those encoding transit peptides, selectable marker proteins, or antisense sequences. These additional promoters may be selected on the basis of the cell type into which the vector will be inserted. Also, promoters which function in bacteria, yeast, and plants are all well taught in the art. The additional promoters may also be selected on the basis of their regulatory features. Examples of such features include enhancement of transcriptional activity, inducibility, tissue specificity, and developmental stage-specificity. Plant functional promoters useful for preferential expression in seed include those from plant storage proteins and from proteins involved in fatty acid biosynthesis in oilseeds. Examples of such promoters include the 5′ regulatory regions from such structural nucleic acid sequences as napin, phaseolin, zein, soybean trypsin inhibitor, ACP, stearoyl-ACP desaturase, and oleosin. Seed-specific regulation is further discussed in EP0255378. Particularly preferred additional promoters in the recombinant vector include the nopaline synthase (nos), mannopine synthase (mas), and octopine synthase (ocs) promoters, which are carried on tumor-inducing plasmids of Agrobacterium tumefaciens; the cauliflower mosaic virus (CaMV) 19S and 35S promoters; the enhanced CaMV 35S promoter; the Figwort Mosaic Virus (FMV) 35S promoter; the light-inducible promoter from the small subunit of ribulose-1,5-bisphosphate carboxylase (ssRUBISCO); the EIF-4A promoter from tobacco. An additional promoter is preferably seed selective, tissue selective, constitutive, or inducible.

The recombinant vector may also contain one or more additional nucleic acid sequences. These additional nucleic acid sequences may generally be any sequences suitable for use in a recombinant vector. Such nucleic acid sequences include, without limitation, any of the nucleic acid sequences, and modified forms thereof, described above. The additional structural nucleic acid sequences may also be operably linked to any of the above described promoters. The one or more structural nucleic acid sequences may each be operably linked to separate promoters. Alternatively, the structural nucleic acid sequences may be operably linked to a single promoter (i.e., a single operon).

The present invention is also directed to transgenic plants and transformed host cells which comprise a promoter operably linked to a heterologous nucleic acid sequence. Other nucleic acid sequences may also be introduced into the plant or host cell along with the promoter and structural nucleic acid sequence. These other sequences may include 3′ transcriptional terminators, 3′ polyadenylation signals, other untranslated nucleic acid sequences, transit or targeting sequences, selectable markers, enhancers, and operators. Preferred nucleic acid sequences of the present invention, including recombinant vectors, structural nucleic acid sequences, promoters, and other regulatory elements, are described above.

The term “transformation” herein refers to the introduction (or transfer) of nucleic acid into a recipient host such as a plant or any plant parts or tissues including plant cells, protoplasts, calli, roots, tubers, seeds, stems, leaves, seedlings, embryos, pollen and microspores. Plants containing the transformed nucleic acid sequence are referred to as “transgenic plants”. Transformed, transgenic and recombinant refer to a host organism such as a plant into which a heterologous nucleic acid molecule (e.g. an expression cassette or a recombinant vector) has been introduced. The nucleic acid can be stably integrated into the genome of the plant.

As used herein, the phrase “transgenic plant” refers to a plant having an introduced nucleic acid stably introduced into a genome of the plant, for example, the nuclear or plastid genomes.

In some embodiments of the present invention, one or more components of a plant, cell, or organism are compared to a plant, cell, or organism having a “similar genetic background.” In a preferred aspect, a “similar genetic background” is a background where the organisms being compared share about 50% or greater of their nuclear genetic material. In a more preferred aspect a similar genetic background is a background where the organisms being compared share about 75% or greater, even more preferably about 90% or greater of their nuclear genetic material. In another even more preferable aspect, a similar genetic background is a background where the organisms being compared are plants, and the plants are isogenic except for any genetic material originally introduced using plant transformation techniques.

A transformed host cell may generally be any cell that is compatible with the present invention. A transformed host plant or cell can be or derived from a monocotyledonous plant or a dicotyledonous plant including, but not limited to canola, maize, mustard, castor bean, sesame, cotton, linseed, soybean, Arabidopsis, Phaseolus, peanut, alfalfa, wheat, rice, oat, sorghum, rapeseed, rye, sugarcane, safflower, oil palms, flax, sunflower, Brassica campestris, Brassica napus, Brassica juncea, Crambe abyssinica. In a particularly preferred embodiment, the plant or cell is or derived from canola. In another particularly preferred embodiment, the plant or cell is or derived from Brassica napus.

A second part of the invention deals with the properties of CYP77A4 for the modification of fatty acids. The most abundant fatty acids in agronomically important plant seeds are fatty acids with a chain length of C16 and C18 possessing between 0 and 3 double bonds in cis configuration. One industrial important modification is the oxygenation of the fatty acid, in particular the end hydroxylation (omega-hydroxylation) of the fatty acid is often desired. The extra addition of an oxygen atom renders the fatty acid more polar but most importantly the extra oxygen atom creates the possibility of forming a covalent linkage with a carboxylic group or with another hydroxyl group of a neighboring fatty acid. In the case the fatty acid is unsaturated (i.e. has double bounds), the oxygenation of these so called unsaturated fatty acids can lead to the formation of an epoxide in the fatty acid structure. An epoxide can be formed on an unsaturated fatty acid in industry by a chemical process (usually with the aid of performic acid) or by an enzymatic process (usually a peroxydase or a desaturase-like enzyme). Epoxy fatty acids not only have the possibility to crosslink but can have interesting physicochemical properties in industry because these fatty acids can be used as stabilizers for PVCs (Metzger and Bornscheuer (2006) Appl. Microbial. Biotechnol 71 (1): 13-22), or they can be used in adhesives and paint (Cahoon et al (2002) Plant Physiol 128 (2): 615-24). One epoxy fatty acid, vernolic acid, is used as a precursor of monomeric components of nylon-11 and nylon-12. During the characterization of the enzymatic function of cytochrome CYP77A4 of Arabidopsis thaliana we have identified a hydroxylase and an epoxidase function of this cytochrome P450 enzyme.

In yet another embodiment the invention provides the use of SEQ ID NO: 2 or a variant or a functional fragment or a functional homologue with a least 85% identity with SEQ ID NO: 2 for the production of epoxy fatty acids in a host cell. In other words the invention provides a process (or method) for the production of epoxy fatty acids in a host cell comprising a) transforming said host cell with a chimeric construct comprising SEQ ID NO: 2 or a variant or a functional fragment or a functional homologue with at least 85% identity with SEQ ID NO: 2 operably linked to at least one suitable regulatory sequence, b) growing the transformed host cells of step a) and c) determining the presence or the absence of epoxy fatty acids in the transformed cells of step b).

In a particular embodiment said host cell is a plant or plant cell.

In yet another particular embodiment said host cell is a yeast cell such as Yarrowia lipolytica, Pichia pastoris, Saccharomyces cerevisiae, a Candida species, a Kluyveromyces species, a Hansenula species, and the like.

In yet another embodiment said epoxy fatty acid is a mono epoxy fatty acid or a bi epoxy fatty acid, or a tri epoxy fatty acid or even a tetra epoxy fatty acid. In a specific embodiment said epoxy fatty acid is vernolic acid.

In yet another embodiment the invention provides a method for the production of 12,13-epoxyoctadeca-9,10-15,16-dienoic acid said method comprising contacting linolenic acid with CYP77A4. In a particular embodiment said production of 12,13-epoxyoctadeca-9,10-15,16-dienoic acid occurs in vivo in plant seeds that comprise linolenic acid. In another particular embodiment said production of 12,13-epoxyoctadeca-9,10-15,16-dienoic acid occurs in vitro through incubation with a source of the CYP77A4 enzyme which can be a purified source or a recombinant source.

In yet another embodiment the invention provides a chimeric construct comprising SEQ ID NO: 2 or a variant or a functional fragment or a functional homologue with at least 85% identity with SEQ ID NO: 2, operably linked to at least one suitable regulatory sequence. In yet another embodiment the invention provides an isolated host cell comprising a chimeric gene of the present invention.

Throughout the description and examples, reference is made to the following sequences represented in the sequence listing:

SEQ ID No 1: nucleotide sequence of the amplified promoter of the CYP77A4 gene of Arabidopsis thaliana

SEQ ID No: 2: sequence of the CYP77A4 gene of Arabidopsis thaliana

SEQ ID No: 3: forward primer for amplification of SEQ ID NO: 1

SEQ ID No: 4: reverse primer for amplification of SEQ ID NO: 1

SEQ ID No: 5: forward primer for amplification of SEQ ID NO: 2

SEQ ID No: 6: reverse primer for amplification of SEQ ID NO: 2

### Materials and General Methods

Unless indicated otherwise, chemicals and reagents in the examples were obtained from Sigma Chemical Company, restriction endonucleases were from Fermentas or Roche-Boehringer, and other modifying enzymes or kits regarding biochemicals and molecular biological assays were from Qiagen, Invitrogen and Q-BIOgene. Bacterial strains were from Invitrogen. The cloning steps carried out for the purposes of the present invention, such as, for example, restriction cleavages, agarose gel electrophoresis, purification of DNA fragments, linking DNA fragments, transformation of E. coli cells, growing bacteria, multiplying phages and sequence analysis of recombinant DNA, are carried out as described by Sambrook (1989). The sequencing of recombinant DNA molecules is carried out using ABI laser fluorescence DNA sequencer following the method of Sanger. Any number of methods well known to those skilled in the art can be used to isolate fragments of a DNA molecule disclosed herein. For example, PCR (polymerase chain reaction) technology can be used to amplify flanking regions from a genomic library of a plant using publicly available sequence information. A number of methods are known to those of skill in the art to amplify unknown DNA sequences adjacent to a core region of known sequence. Methods include but are not limited to inverse PCR, vectorette PCR, Y-shaped PCR, and genome walking approaches. DNA molecule fragments can also be obtained by other techniques such as by directly synthesizing the fragment by chemical means, as is commonly practiced by using an automated oligonucleotide synthesizer. For the present invention, the DNA molecules were isolated by designing PCR primers based on available sequence information. Standard materials and methods for plant molecular work are described in Plant Molecular Biology Labfax (1993) by R. D. D. Croy, jointly published by BIOS Scientific Publications Ltd (UK) and Blackwell Scientific Publications.

Chemicals—Radiolabeled [1-¹⁴C]lauric acid (45 Ci/mol) was from CEA (Gif sur Yvette, France) [1-¹⁴C]oleic acid (50 Ci/mol), [1-¹⁴C]linoleic acid (58 Ci/mol), and [1-¹⁴C]α-linolenic acid (52 Ci/mol) were from Perkin Elmer Life Sciences. Linolelaidic acid was from Sigma. The silylating reagent N,O-bistrimethylsilyltrifluoroacetamide containing 1% of trimethylchlorosilane was from Pierce (Rockfold, Ill.). NADPH was from Sigma (Saint Louis, Mo.). Thin layer plates (Silica Gel G60 F254; 0.25 mm) were from Merck (Darmstadt, Germany).

Heterologous expression of CYP77A4 in Yeast—For functional expression of the full length CYP77A4 clone, we used a yeast expression system specifically developed for the expression of P450 enzymes and consisting of plasmid pYeDP60 and Saccharomyces cerevisiae WAT11 strain (Pompon D. et al (1996) Methods Enzymol 272, 51-64. Yeast cultures were grown and CYP77A4 expression was induced as described in Pompon et al. (1996) see above from one isolated transformed colony. After growth, cells were harvested by centrifugation and manually broken with glass beads (0.45 mm diameter) in 50 mM Tris-HCl buffer (pH 7.5) containing 1 mM EDTA and 600 mM sorbitol. The homogenate was centrifuged for 10 min at 10,000 g. The resulting supernatant was centrifuged for 1 h at 100,000 g. The pellet consisting of microsomal membranes was resuspended in 50 mM Tris-HCl (pH 7.4), 1 mM EDTA and 30% (v/v) glycerol with a Potter-Elvehjem homogenizer and stored at −30° C. The volume of resuspension buffer is proportional to the weight of yeast pellet: microsomes extracted from 6 g of yeast are resuspended in 3 ml of buffer. All procedures for microsomal preparation were carried out at 0-4° C. The cytochrome P450 content was measured by the method of Omura and Sato (1964) J Biol Chem 239, 2370-2378.

Plant material and microsomal preparation—After sterilization, Arabidopsis (ecotype Col-0) seeds were grown on Murashige and Skoog medium (MS medium 4.2 g/l, sucrose 10 g/l, pastagar B 8 g/l, myoinositol 100 mg/l, thiamine 10 mg/l, nicotinic acid 1 mg/l and pyridoxine 1 mg/l—final pH 5.7) during five weeks. Arabidopsis plants (approximately 10 g) were homogenized with mortar and pestle in 50 ml of extraction buffer (250 mM tricine, 50 mM NaHSO₃, 5 g/l bovine serum albumin, 2 mM EDTA, 100 mM ascorbic acid and 2 mM dithiothreitol—final pH 8.2). The homogenate was filtered through 50 μm nylon filtration cloth and centrifuged for 10 min at 10,000 g. The resulting supernatant was centrifuged for 1 h at 100,000 g. The supernatant (cytosol) was directly stored at −30° C. and the microsomal pellet was resuspended in the buffer at pH 8.2 (50 mM NaCl, 100 mM tricine, 250 mM sucrose, 2 mM EDTA and 2 mM dithiothreitol), with a Potter-Elvehjem homogenizer and stored at −30° C. All procedures for microsomal preparation were carried out at 0-4° C.

Enzyme activities—All radiolabeled substrates were dissolved in ethanol that was evaporated before the addition of microsomes into the glass tube. Resolubilization of the substrates was confirmed by measuring the radioactivity of the incubation media. Enzymatic activities of CYP77A4 from transformed yeast or Arabidopsis microsomes were determined by following the formation rate of metabolites. The standard assay (0.1 ml) contained 20 mM sodium phosphate (pH 7.4), 1 mM NADPH, and radiolabeled substrate (100 μM). The reaction was initiated by the addition of NADPH and was stopped by the addition of 20 μl acetonitrile (containing 0.2% acetic acid). The reaction products were resolved by TLC or HPLC as described below. For kinetic studies we incubated 4.5 pmoles of CYP77A4 during 5 min with various concentrations of substrate ranging from 10 to 200 μM for C_{12:0}, from 5 to 120 μM for C_{18:1}, from 5 to 80 μM for C_{18:2} and from 5 to 120 μM for C_{18:3.}

Thin layer chromatographic (TLC) methods—Incubation media were directly spotted on TLC plates. For separation of metabolites from residual substrate, TLC were developed with a mixture of diethyl ether/light petroleum (boiling point, 40-60° C.)/formic acid (50:50:1, v/v/v). The plates were scanned with a radioactivity detector (Raytest Rita Star). The area corresponding to the metabolites were scraped into counting vials and quantified by liquid scintillation, or they were eluted from the silica with 10 ml of diethyl ether, which was removed by evaporation. They were then subjected to GC/MS analysis.

GC/MS analysis—GC-MS analysis were carried out on a gas chromatograph (Agilent 6890 Series) equipped with a 30-m capillary column with an internal diameter of 0.25 mm and a film thickness of 0.25 μm (HP-5MS). The gas chromatograph was combined with a quadrupole mass selective detector (Agilent 5973N). Mass spectra were recorded at 70 eV and analysed as in Eglinton et al. (1968) Org. Mass. Spectrom 1, 593-611.

### EXAMPLES

### 1. PCR Quantification of CYP77A4 Expression in Arabidopsis thaliana

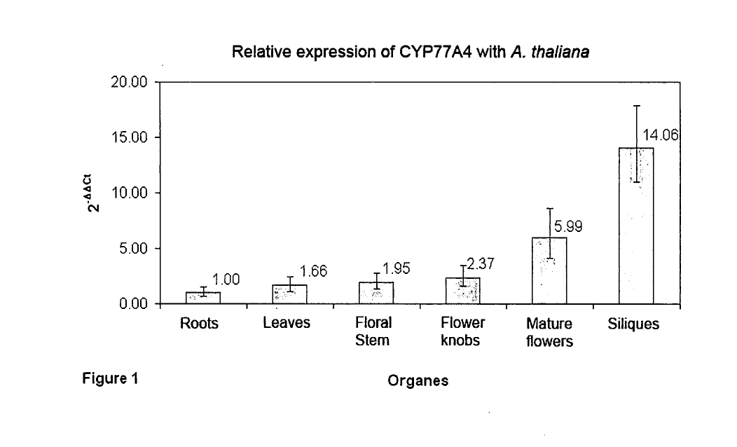

RNA was isolated from different organs (roots, leaves, floral stern, flower buds, flowers and siliques) of a mature Arabidopsis thaliana plant and cDNA was generated by RT-PCR. The relative expression of CYP77A4 (with respect to the ubiquitous and constitutive expression of actine 2) was determined with the SYBR Green quantitative PCR protocol (qPCR) as described in detail by Ruiz-Ruiz S. et al (2007) J. Virol. Methods 145 (2):96-105 was carried out with the GeneAmp 5700 Sequence Detection System (Applied Biosystems) The expression of CYP77A4 is normalized in all the organs with respect to the internal reference gene (actine 2 of Arabidopsis thaliana, GenBank locus At5g09810). FIG. 1 shows the details of the qPCR analysis of the different organs. As can be seen in FIG. 1 highest expression is found in the siliques (14 fold higher than the faintest value of expression in the roots which was set as value 1) and in the mature flowers (6 fold higher than in roots).

### 2. Generation of an Expression Cassette Comprising a Seed Preferred Promoter of Arabidopsis thaliana

To isolate the promoter fragment described by SEQ ID NO: 1 genomic DNA is isolated from Arabidopsis thaliana (ecotype Columbia), as described (Galblati M et al (2000) Funct. Integr. Genomics 1 (1):25-34. The isolated genomic DNA is employed as matrix DNA for a polymerase chain reaction (PCR) mediated amplification using the oligonucleotide primers and protocols indicated below.

Amplification is Carried Out as Follows:

10 ng genomic DNA of Arabidopsis thaliana

1×PCR buffer

1.5 mM MgCl₂,

200 μM each of dATP, dCTP, dGTP und dTTP

10 pmol of each oligonucleotide primers:

Forward:
(SEQ ID No: 3)
5′_GGGGACAAGTTTGTACAAAAAAGCAGGCTCTAAGAGTTTTGCTTT
GGTCTATTG_3′
Reverse:
(SEQ ID No: 4)
5′_GGGGACCACTTTGTACAAGAAAGCTGGGTTTTAGCTCTGTTTATT
TCTTGTTG_3′

3.5 Units High Fidelity polymerase (Roche-Boehringer) in a final volume of 50 μl

The following temperature program was employed for the various amplifications (BIORAD Thermocycler).

1. 96° C. for 10 min

2. 58° C. for 2 min, followed by 72° C. for 3 min and 96° C. for 3 min. Repeated 30 times.

3. 58° C. for 1 min, followed by 72° C. for 10 min.

4. Storage at 4° C.

Sequence verification of the PCR product resulted in the nucleotide sequence as depicted in SEQ ID NO: 1. SEQ ID NO: 1 has an extra Adenine (A) inserted at position 514 and in position 1497 Cytidine (C) replaces Thymidine (T) with respect to the promoter sequence of the GenBank A. thaliana locus At5g04660 (position 1334549 to position 1336049 on Chromosome V, note that the CYP77A4 open reading frame starts at position 1336050)

The resulting PCR-product, adapted to the Gateway cloning technology, was inserted into the pDONR207 gateway entry vector (invitrogen) by an overnight incubation at 25° C. of 60 ng of the PCR product with 75 ng of pDONR207 and 1 μl of BP clonase II (Invitrogen) in a final volume of 5 μl. The sequence of interest was then transferred in the destination vector pBGWFS7.0 by an overnight incubation at 25° C. of 500 ng of the recombinant pDONR207 with 500 ng of pBGWFS7.0 and 1 μl of the LR clonase II (Invitrogen) in a final volume of 5 μl. Thus, a CYP77A4-promoter (1502 base pairs situated upstream of the startcodon of the CYP77A4 sequence)—GUS (a gene encoding beta-glucuronidase) construct is an expression cassette which was cloned in the plant transformation vector pBGWFS7.0.

3. Expression Profile of the CYP77A4 Promoter:GUS Construct in Stably Transformed Arabidopsis thaliana Plants

In a next step the recombinant vector comprising the expression cassette of example 2 was used to stably transform Arabidopsis thaliana. The protocol for Agrobacterium mediated transformation of A. thaliana was according to the floral dip method described by Clough S J and Bent A F (1998) Plant J 16(6): 735-43. β-glucuronidase activity was monitored in planta with the chromogenic substrate X-Gluc (5-bromo-4-Chloro-3-indolyl-β-D-glucuronic acid) during corresponding activity assays (Jefferson R A et al (1987) EMBO J. 20; 6(13):3901-7). For determination of promoter activity and tissue specificity plant tissue is dissected, embedded, stained and analyzed as described (e.g., Bäumlein H et al (1991) Mol. Gen. Genetics 225 (3):459-67). Thus, the activity of beta-glucuronidase in the transformed plants was witnessed by the presence of the blue color due to the enzymatic metabolism of the substrate X-Gluc.

In a first experiment young plantlets (1 month) of transformed A. thaliana plants were used. After an incubation of the plants for 1 night at 37° C. with a solution comprising X-gluc and rinsing the plants afterwards with ethanol there was a strong blue signal in trichomes of the plants. Remarkably this signal was strongest in the youngest trichomes.

In a second experiment adult plants or transformed A. thaliana plants, possessing inflorescences at different stages of maturity, were investigated for GUS expression. GUS expression was localized at the floral buds, mature flowers and developing seeds. Especially in seed, GUS expression is detected in non mature seed where the fruit is still green, closed and the seeds are filled with water. The tegument (or seed coat) does not show any GUS staining. The embryo shows also a high GUS staining.

### 4. Induction of the Expression of CYP77A4

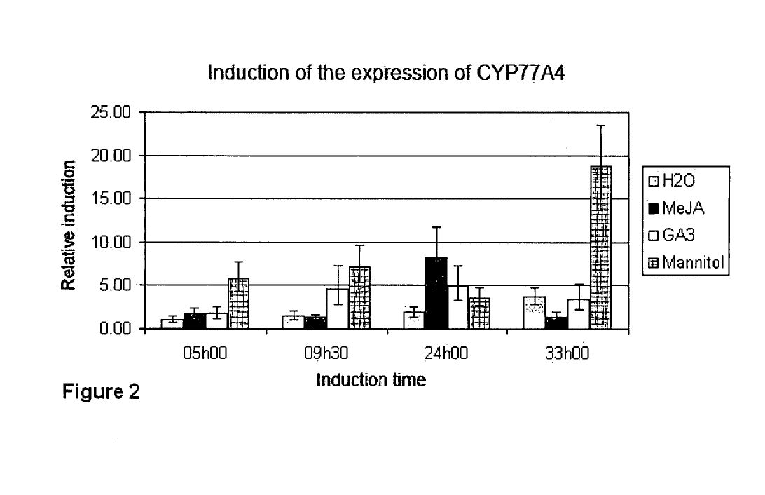

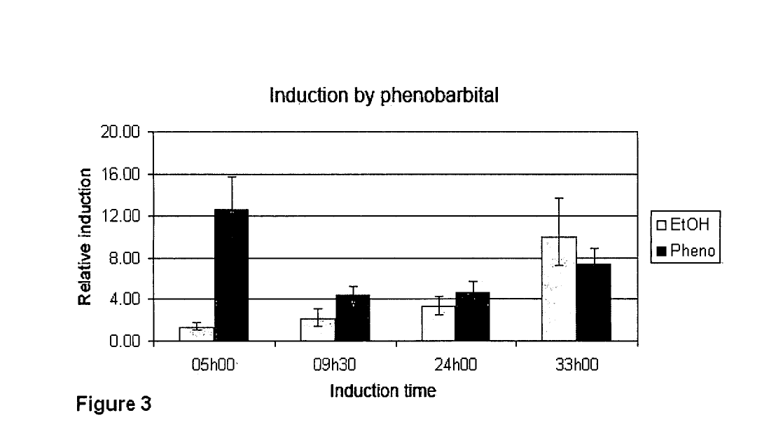

Beside the preferred expression in seeds of the cytochrome CYP77A4 under normal growth conditions we also investigated if this cytochrome could be induced by the addition of external factors such as methyljasmonate (MeJA), gibberellin (GA3), mannitol and phenobarbital (see FIGS. 2 and 3). Remarkably mannitol induced the expression of CYP77A4 of almost 20-fold. Phenobarbital was applied because it is known to induce cytochrome P450 enzymes capable of modifying fatty acids in animal systems. Indeed, after only 5 hours after the application of phenobarbital it was observed that the expression of CYP77A4 was enhanced by almost 12-fold. Measurements of the CYP77A4 expression were carried out via quantitative PCR as outlined in example 1. RNA extractions have been performed on entire three weeks old young plantlets grown on sterile conditions in MS medium.

### 5. Vector Construction for Overexpression and Gene “Knockout” Experiments

Vectors used for expression of full-length “candidate nucleic acids” of interest in plants are designed to (over)express the protein of interest in a seed-preferred way and are of two general types, biolistic and binary, depending on the plant transformation method to be used. For biolistic transformation (with so called biolistic vectors), the requirements are as follows.

A backbone with a bacterial selectable marker (typically an antibiotic resistance gene) and origin of replication functional in Escherichia coli (e.g. ColEI), and (2) a plant-specific portion consisting of: a. a gene expression cassette consisting of the promoter depicted in SEQ ID NO: 1 or a functional fragment of at least 50 consecutive bases of SEQ ID NO: 1 or a nucleotide sequence having at least 40% identity with SEQ ID NO; 1 or a functional fragment of at least 40% identity to a fragment of at least 50 consecutive bases of SEQ ID NO: 1—the gene of interest (typically a full-length cDNA) and a transcriptional terminator (e.g. the Agrobacterium tumefaciens nos terminator); b. a plant selectable marker cassette, consisting of a suitable promoter, a selectable marker gene and transcriptional terminator (e.g. the nos terminator).

Vectors designed for transformation by Agrobacterium tumefaciens (so called binary vectors) consist of a backbone with a bacterial selectable marker functional in both E. coli and A. tumefaciens (e.g, spectinomycin resistance mediated by the aadA gene) and two origins of replication, functional in each of aforementioned bacterial hosts, plus the A. tumefaciens virG gene, (2) a plant-specific portion as described for biolistic vectors above, except in this instance this portion is flanked by A. tumefaciens right and left border sequences which mediate transfer of the DNA flanked by these two sequences to the plant.

Vectors designed for reducing or abolishing expression of a single gene or of a family or related genes (such as gene silencing vectors) preferentially in seeds are also of two general types corresponding to the methodology used to downregulate gene expression: antisense or double-stranded RNA interference (dsRNAi). For antisense vectors, a full-length or partial gene fragment (typically, a portion of the cDNA) can be used in the same vectors described for full-length expression, as part of the gene expression cassette. For antisense-mediated down-regulation of gene expression, the coding region of the gene or gene fragment will be in the opposite orientation relative to the promoter whereby mRNA will be made from the non-coding (antisense) strand in a seed-preferred way in planta.

For dsRNAi vectors, a partial gene fragment is used in the gene expression cassette, and is expressed in both the sense and antisense orientations, separated by a spacer region (typically a plant intron or a selectable marker). Vectors of this type are designed to form a double-stranded mRNA stern, resulting from the base pairing of the two complementary gene fragments in a seed-preferred way in planta. Biolistic or binary vectors designed for overexpression or knockout can vary in a number of different ways, including eg. the selectable markers used in plant and bacteria, the transcriptional terminators used in the gene expression and plant selectable marker cassettes, and the methodologies used for cloning in gene or gene fragments of interest (typically, conventional restriction enzyme-mediated or Gateway™ recombinase-based cloning).

### 6. Recombinant Expression of CYC77A4 in the Yeast Saccharomyces cerevisiae

The coding sequence of CYP77A4 (GenBank locus At5g04660, depicted in SEQ ID NO 2) was subcloned by PCR on Arabidopsis thaliana Col0 genomic DNA into the pCRII-Topo (Invitrogen) vector. Primers used (Forward: 5′_CCCCAGATCTATGTTTCCTCTAATCTC_{—}3′ (SEQ ID No: 5)/Reverse: 5′_GGGGGGTACCCTAAATCCTTGGTTTG_{—}3′ (SEQ ID No: 6) for amplification of the coding sequences allowed us to insert the Bgl-II and Kpn-I restriction sites respectively at the 5′ and 3′ extremity of the coding sequence of CYP77A4. After sequence verification, a subcloning of SEQ ID NO 2 was carried out into the S. cerevisiae expression vector pYeDP60 (after digestion of pYeDP60 by BamHI and Kpn-I which generate sticky ends compatible with Bgl-II and Kpn-I digested, amplified SEQ ID No: 2) thereby rendering SEQ ID No 2 under expression control of the inducible yeast chimeric GAL10-CYC1 hybrid promoter (Pompon, D et al (1996) Methods Enzymol 272: 51-64). The yeast strain WAT11 (Pompon, D et al (1996) as above) was subsequently transformed with the recombinant pYeDP60 vector. Expression of SEQ ID NO: 2 was induced by the addition of 2% galactose to the culture medium of a recombinant yeast comprising pYeDP60:CYP77A4 and after the induction phase microsomes were prepared. The expression of CYP77A4 in a solution of microsomes was found to be 4 μM (or about 400 pmol/ml).

### 7. Substrate Specificity of Recombinant CYP74A4

A range of saturated and insaturated fatty acids, with a chain length varying between C-10 and C-18, were tested as an in vitro substrate for modification with recombinant CYP74A4 (present in the microsome fraction of the recombinant yeast strain). Microsomes were prepared according to Bak et al (2000) Plant Physiology 123 (4):1437-48. After incubation of the substrates the resulting metabolites were tested with thin layer chromatography (TLC) or gas chromatography coupled with a mass spectrum analyzer (GC-MS). Radioactive labeled fatty acids (¹⁴COOH-labeled as purchased with American Radiolabeled Chemicals, Inc) were used for TLC analysis. For GC-analysis the fatty acids were derivatized towards the methylic ester with the aid of diazomethane to make them more volatile. Hydroxylated groups of the fatty acids were modified with MSTFA (Thermo-scientific) before mass spectrometric analysis, MSTA adds a trimethylsilyl group on an existing hydroxyl-group and makes the derivatized fatty acid more “fragile” under mass spectrometric conditions. Since epoxy-groups cannot be derivatized by MSTA they had to be opened chemically before derivatization. One method for opening an epoxide is by using sulfuric acid in methanol as described by Cahoon et al (2002) Plant Physiol 128 (2): 615-24.

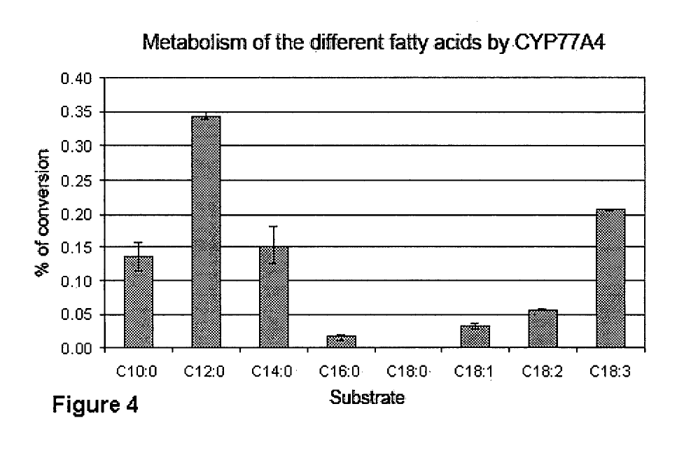

FIG. 4 shows the conversion of the different fatty acids by CYP74A4. It is apparent from this FIG. 4 that C12:0 is the optimal substrate for CYP77A4. GC-MS analysis of this fraction showed that 5 different hydroxylated metabolites were generated of the C12:0 fatty acid (lauric acid) (the omega-1 (53%), omega-2 (15.2%), omega-3 (7.6%), omega-4 (18.2%) and omega-5 (6%)). Thus, the terminal (omega-position) position of the C12:0 fatty acid was not a preferred substrate for hydroxylation and the dominant hydroxylated position was found at the omega-1 position. Remarkably, no modification could be detected for C18:0 (stearic acid) but an increasing order of conversion was found for C18:1 (oleic acid), C18:2 (linoleic acid) and C18:3 (linolenic acid).

### 8. Mono-Epoxidation of Oleic Acid, Linoleic Acid and Linolenic Acid

100 μM of radioactive labeled oleic acid (C18:1) was incubated during 15 minutes at 27° C. with 10 pmole CYP77A4 (the recombinant microsome fraction) in the presence of NADPH. Two peaks were detected in thin layer chromatography. The first peak (peak 1) had the expected mobility of hydroxylated derivatives of oleic acid. The second peak (peak 2) had an unexpected mobility because of its intermediate polarity between the substrate and the hydroxylated derivatives (peak 1). Peak 2 was subsequently analyzed by GC-MS analysis. The mass spectrum of one of the structures in peak 2 was found to be identical to the mass spectrum of the epoxy form (at position 9, 10) of oleic acid (this epoxy form is also called 9,10-epoxystearic acid). The calculated kinetic parameters for the metabolism of oleic acid by CYP77A4 are Kₘ of 87±23 μM and Vₘₐₓ of 26±5 nmol·min⁻¹·nmol⁻¹ P450. These parameters were calculated with the program “Enzyme Kinetics” which is available in Sigma Plot.

100 μM of linoleic acid (C18:2) was incubated during 15 minutes at 27° C. with 10 pmole of CYP77A4 (the recombinant microsome fraction) in the presence of NADPH. The thin layer chromatography indicated the presence of a mono-epoxygenated linoleic acid but the presence of the other two cis double bounds (9-10 and 12-13) in linoleic acid could give rise to several possibilities for epoxidation (the presence of 9-10 epoxidation, the presence of 12-13 epoxidation or a mixture of the two epoxy products). A further analysis by GC-MS analysis of the suspected mono-epoxy product(s) indicated that the only product formed was the 12-13 epoxy form of linolenic acid (also known in the art as vernolic acid). The calculated kinetic parameters for the metabolism of linoleic acid by CYP77A4 are: Kₘ (Michaelis Menten constant) of 61±3 μM and Vₘₐₓ of 13±0.3 nmol·min⁻¹·nmol⁻¹ P450.

100 μM of linolenic acid (C18:3) was incubated during 15 minutes at 27° C. with 10 pmole of CYP77A4 (the recombinant microsome fraction) in the presence of NADPH. Again the thin layer chromatography indicated the presence of a mono-epoxy form of linolenic acid. Because of the presence of three double bounds in linolenic acid three different mono-epoxy forms could be formed. It was found that the 12-13 mono-epoxy form of linolenic acid was the predominant structure (87%), the structure is designated as 12,13-epoxyoctadeca-9,10-15,16-dienoic acid. The other two mono-epoxy structures were identified at each 6.5%. The calculated kinetic parameters for the metabolism of linolenic acid by CYP77A4 are: Kₘ of 29±4 μM and Vₘₐₓ of 38±23 nmol·min⁻¹·nmol⁻¹ P450.

### 9. Di-Epoxydation of Vernolic Acid

In a next step the capacity of CYP77A4 was tested for the formation of a di-epoxide of an unsaturated fatty acid. Thereto a commercially available mono-epoxide, vernolic acid, was incubated as a substrate with CYP77A4. 100 μM of radioactively labeled vernolic acid was incubated for 20 min at 27° C. with 10 pmole of CYCP77A4 in the presence or in the absence of NADPH. The thin layer chromatogram showed a novel peak which could correspond with a di-epoxy form of vernolic acid. Indeed, the more detailed GC-MS analysis of this peak clearly showed that a di-epoxide derivative of linoleic acid was generated by CYP77A4.

1. An expression cassette for regulating seed-preferential expression in plants comprising a promoter operably linked to a nucleic acid which is heterologous in relation to said promoter and wherein said promoter is selected from the group consisting of (a) SEQ ID NO 1 or a variant thereof, (b) a functional fragment of at least 50 consecutive bases of SEQ ID NO 1 having promoter activity, (c) a nucleotide sequence with at least 40% identity with SEQ ID NO 1 or the complement thereof and (d) a nucleotide sequence hybridizing under conditions equivalent to hybridization in 7% sodium dodecyl sulfate, 0.5 M NaPO₄, 1 mM EDTA at 50° C. with washing in 2×SSC, 0.1% SDS at 50° C. to a nucleotide sequence depicted in SEQ ID NO: 1 or a fragment of at least 50 consecutive bases of SEQ ID NO: 1 or the complement thereof.

2. The expression cassette of claim 1 wherein expression of the nucleic acid results in expression of a protein, or expression of an antisense RNA, sense or double-stranded RNA.

3. A recombinant vector comprising an expression cassette according to claim 1.

4. A transformed plant containing the expression cassette of claim 1.

5. The transformed plant of claim 4 wherein said transformed plant is from a plant used for oil production.

6. The transformed plant according to claim 5 wherein the plant is selected from the group consisting of canola, maize, mustard, castor bean, sesame, cotton, linseed, soybean, Arabidopsis, Phaseolus, peanut, alfalfa, wheat, rice, oat, sorghum, rapeseed, rye, sugarcane, safflower, oil palms, flax, sunflower, Brassica campestris, Brassica napus, Brassica juncea and Crambe abyssinica.

7. A plant cell comprising an expression cassette of claim 1.

8. A microspore comprising an expression cassette of claim 1.

9. A seed generated from a transformed plant according to claim 4 wherein the seed comprises an expression cassette for regulating seed-preferential expression in plants comprising a promoter operably linked to a nucleic acid which is heterologous in relation to said promoter and wherein said promoter is selected from the group consisting of (a) SEQ ID NO 1 or a variant thereof, (b) a functional fragment of at least 50 consecutive bases of SEQ ID NO 1 having promoter activity, (c) a nucleotide sequence with at least 40% identity with SEQ ID NO 1 or the complement thereof and (d) a nucleotide sequence hybridizing under conditions equivalent to hybridization in 7% sodium dodecyl sulfate, 0.5 M NaPO₄, 1 mM EDTA at 50° C. with washing in 2×SSC, 0.1% SDS at 50° C. to a nucleotide sequence depicted in SEQ ID NO: 1 or a fragment of at least 50 consecutive bases of SEQ ID NO: 1 or the complement thereof.

10. A method of producing a transformed plant according to claim 4 comprising (a) providing an expression cassette of for regulating seed-preferential expression in plants comprising a promoter operably linked to a nucleic acid which is heterologous in relation to said promoter and wherein said promoter is selected from the group consisting of (a) SEQ ID NO 1 or a variant thereof, (b) a functional fragment of at least 50 consecutive bases of SEQ ID NO 1 having promoter activity, (c) a nucleotide sequence with at least 40% identity with SEQ ID NO 1 or the complement thereof and (d) a nucleotide sequence hybridizing under conditions equivalent to hybridization in 7% sodium dodecyl sulfate, 0.5 M NaPO₄, 1 mM EDTA at 50° C. with washing in 2×SSC, 0.1% SDS at 50° C. to a nucleotide sequence depicted in SEQ ID NO: 1 or a fragment of at least 50 consecutive bases of SEQ ID NO: 1 or the complement thereof and (b) transforming a plant with said expression cassette.

11. A method of producing a seed comprising a nucleic acid of claim 1 comprising (a) growing a transformed plant containing the expression vector of claim 1, wherein said transformed plant produces said seed and said nucleic acid is transcribed in said seed, and (b) isolating said seed from said transformed plant.
